(19) United States
(12) Patent Application Publication
(10) Pub. No.: US 2009/0130017 A1
(43) Pub. Date: May 21, 2009

(54) Targeted short-lived drug delivery

(75) Inventors: Paul G. Allen, Seattle, WA (US); Edward S. Boyden, Cambridge, MA (US); Roderick A. Hyde, Redmond, WA (US); Muriel Y. Ishikawa, Livermore, CA (US); Stephen L. Malaska, Redmond, WA (US); Nathan P. Myhrvold, Medina, WA (US); Clarence T. Tegreene, Bellevue, WA (US); Lowell L. Wood, JR., Bellevue, WA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC

(21) Appl. No.: 12/290,457

(22) Filed: Oct. 29, 2008

Related U.S. Application Data
(60) Provisional application No. 61/124,822, filed on Nov. 19, 2007.

(51) Int. Cl. A61K 51/00; A61K 38/49; A61K 31/727; A61K 31/7105; A61K 31/137; A61K 31/7048; A61K 31/40

(52) U.S. Cl. 424/1.11; 424/94.64; 514/56; 514/44; 514/649; 514/653; 514/29; 514/423; 424/422

(57) ABSTRACT

An aspect of the disclosure includes a system for delivering therapeutic agents. In an embodiment, the system includes an implantable medical device comprising at least one reservoir that holds at least one therapeutic agent. Additionally the device includes a delivery mechanism that provides non-systemic in vivo delivery of the at least one therapeutic agent to a local area of an animal in a therapeutically-effective concentration, wherein the therapeutically-effective concentration is in excess of a concentration that would produce a toxic effect when administered systemically to the animal. Furthermore, the at least one therapeutic agent has short half-life. A further aspect of the disclosure includes a method of delivering a therapeutic agent in vivo at non-systemic high doses to a localized area of an animal.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)). All subject matter of the Related Applications and of any and all parent, grandparent, great-grandparent, etc. applications of the Related Applications is incorporated herein by reference to the extent such subject matter is not inconsistent herewith.

### RELATED APPLICATIONS

For purposes of the USPTO extra-statutory requirements, the present application constitutes a continuation-in-part of U.S. Patent Application No. 61/124,822, entitled TARGETED SHORT-LIVED DRUG DELIVERY, naming Paul G. Allen, Edward S. Boyden, William H. Gates, III, Roderick A. Hyde, Muriel Y. Ishikawa, Edward K. Y. Jung, Eric C. Leuthardt, Stephen L. Malaska, Nathan P. Myhrvold, Dennis J. Rivet, Michael A. Smith, Clarence T. Tegreene, Lowell L. Wood, Jr., and Victoria Y. H. Wood, as inventors, filed 19 Nov. 2007, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### TECHNICAL FIELD

The present application relates, in general, to devices, methods or systems for treatment or management of disease, disorders, or conditions, using non-systemic delivery of therapeutic agents.

### SUMMARY

Many therapeutic agents are administered systemically. The formulation of many therapeutic agents is normally designed to require that the therapeutic agents have adequate exposure characteristics, inter alia, long half-lives. That is, a therapeutic agent must be able to get to its target, and must not quickly be eliminated, metabolized, bound to a macromolecule or otherwise neutralized, and that the therapeutic agent must be able to arrive at a target in sufficient concentration to produce a therapeutic effect. In order to overcome certain liabilities of some therapeutic agents, a higher dose of a therapeutic agent may be administered. Higher systemic concentrations can lead to toxicity, thereby reducing a “therapeutic window” of a therapeutic agent.

This disclosure relates to using non-systemic doses of therapeutic agents to address local areas in an animal body, using short-lived therapeutic agents in order to minimize toxicity. The systems, devices, therapeutic agents and methods described and claimed herein deliver therapeutic agents to a local area in excess of a therapeutically-effective systemic concentration, while reducing systemic toxic effects, due to the short half-life of the therapeutic agent.

An embodiment of the disclosure includes a system for delivering therapeutic agents. In an embodiment, the system includes a medical device comprising at least one reservoir that holds at least one therapeutic agent. Additionally, the device includes a delivery mechanism configured to provide non-systemic delivery of the at least one therapeutic agent to a local area of an animal in a therapeutically-effective concentration, wherein the therapeutically-effective concentration is in excess of a concentration that would produce a toxic effect when administered systemically to the animal. The medical device may be implantable. Implantable devices include devices that reside, either entirely or partially, temporarily or permanently in vivo; or includes devices that may have a component that reside temporarily or permanently in vivo. Furthermore, the at least one therapeutic agent includes agents that have a short half-life.

In an embodiment, the at least one therapeutic agent has a half-life that is less than about 5 hours. In an embodiment, the at least one therapeutic agent has a half-life that is less than about 4 hours. In an embodiment, the at least one therapeutic agent has a half-life that is less than about 3 hours. In an embodiment, the at least one therapeutic agent has a half-life that less than about 2 hours. In an embodiment, the at least one therapeutic agent has a half-life less than about 1 hour.

An embodiment calls for the at least one therapeutic agent to include an anti-coagulant of blood. In an embodiment, an anti-coagulant of blood may include, for example, without limitation, tissue plasminogen activator or Streptokinase or heparin.

In an embodiment, at least one therapeutic agent may include an anti-cancer agent. In an embodiment, anti-cancer agents may include, for example, without limitation, at least one of the following agents: podophyllotoxin, etoposide, chlorambucil, cisplatin, oxaliplatin or carboplatin. In an embodiment provides that anti-cancer agents may include the modified oligonucleotide OGX-011.

An embodiment of an implantable medical device comprises at least one therapeutic agent that may include at least one anti-inflammatory agent. Anti-inflammatory agents may include, for example, without limitation, at least one of an adenosine, granulocyte-macrophage colony stimulating factors (GM-CSF) or anti-GM-CSF scFv or antibodies that neutralize GM-CSF or Fab′ fragments of the antibodies.

In an embodiment, an implantable medical device includes at least one therapeutic agent, which may include at least one agent for gene therapy of diseases. The at least one therapeutic agent may include, for example, without limitation, at least one of an RNA or a DNA. In some embodiments, the RNAs may include siRNA or RNAi. In an embodiment, the DNAs may include nucleotide sequences of nerve growth factors.

In an embodiment, an implantable medical device comprises at least one therapeutic agent that may include at least one beta blocker. The at least one beta blocker may include, for example, without limitation, at least one of the following therapeutic agents: esmolol, propranolol, atenolol, oxprenolol, alprenolol, prindolol, timolol, acebutalol, metaprolol or nadolol.

In an embodiment, an implantable medical device comprises at least one therapeutic agent that may include at least one agent to treat cardiac arrhythmias. The at least one agent to treat cardiac arrhythmias may include, for example, without limitation, at least one of lidocaine, atropine or adenosine.

In an embodiment, an implantable medical device comprises at least one therapeutic agent that may include at least one agent to treat diabetes. These therapeutic agents include, for example, without limitation, glucagon or glucagon-like peptide-1.

In an embodiment, an implantable medical device comprises at least one therapeutic agent that may include at least one agent to treat neurological or behavioral disorders. The at least one agent to treat neurological or behavioral disorders include, for example, without limitation, venlafaxine.

In an embodiment, an implantable medical device comprises at least one therapeutic agent to treat animal airway passages, such as albuterol.

In an embodiment, an implantable medical device comprises at least one therapeutic agent that may include at least one antibiotic agent. The at least one antibiotic agent may include erythromycin or its derivatives.

In an embodiment, an implantable medical device comprises at least one therapeutic agent that may include at least one cholesterol-lowering therapeutic agent. In an embodiment, the at least one cholesterol-lowering therapeutic agent may include, for example, without limitation, atorvastatin.

In an embodiment, an implantable medical device comprises at least one of a catheter, a radioactive seed, a stent, a needle, a syringe, a trocar, a microchip or a tube. Furthermore, the implantable device may includes at least one reservoir, which may include, for example, without limitation, one of a lipid bilayer, a microcapsule, a liposome, a granule, capsule, a catheter, a radioactive seed, a stent, a needle or a tube.

An embodiment includes a method of delivering in vivoin vivo at least one therapeutic agent to an animal, which comprises: providing non-systemic delivery of the at least one therapeutic agent at a therapeutically-effective concentration to a local area of the animal, wherein the therapeutically-effective concentration of the therapeutic agent is in excess of a concentration that would produce a toxic effect when administered systemically to the animal; and wherein the delivering the at least one therapeutic agent that has a short half-life. In an embodiment, a non-systemic delivery of the at least one therapeutic agent to a local area of the animal includes delivery to, for example, at least one of a joint, a heart, a kidney, a lung, a brain, a liver, a spleen, a blood vessel, a vein, a capillary, a stomach, an intestine, a duodenum, a fallopian tube, a limb, a non-vasculaturized area or a bone. In a further embodiment, the therapeutically-effective concentration of the at least one therapeutic agent includes a concentration that constitutes sufficient effect, by way of non-limiting that produces a healing effect, that produces an ameliorating effect, that produces a reduction in pain, that results in a reduction in tumors, that produces a reduction in blood pressure, results in an increase in blood pressure or provides an improvement in management of a diseased state.

In an embodiment, a method of delivering in vivo therapeutic agents includes delivering at least one therapeutic agent that has half-lives not longer than 5 hours.

Another embodiment of a method of delivering in vivo therapeutic agents calls for at least one therapeutic agent to include an anti-coagulant of blood. In an embodiment, the anti-coagulants of blood may include, for example, without limitation, tissue plasminogen activator or Streptokinase or heparin.

In still another embodiment, a method of delivering therapeutic agents in vivo includes delivering at least one therapeutic agent that may include anti-cancer agents. In an embodiment, the anti-cancer agents may include, for example, without limitation, at least one of the following agents: podophyllotoxin, etoposide, chlorambucil, cisplatin, oxaliplatin or carboplatin. Another embodiment provides that the anti-cancer agents may include the modified oligonucleotide OGX-011.

An embodiment of a method of delivering therapeutic agents comprises at least one therapeutic agent that may include anti-inflammatory agents. The anti-inflammatory agents may include, for example, without limitation, at least one of an adenosine, granulocyte-macrophage colony stimulating factors (GM-CSF) or anti-GM-CSF scFv or antibodies that neutralize GM-CSF or Fab′ fragments of the antibodies.

In an embodiment, a method of delivering therapeutic agents includes at least one therapeutic agent, which may include at least one agent for gene therapy of diseases. The at least one therapeutic agent may include, for example, without limitation, at least one of an RNA or a DNA. In some embodiments, the RNAs may include siRNA or RNAi. In an embodiment, the DNAs may include nucleotide sequences of nerve growth factors.

In an embodiment, a method of delivering therapeutic agents comprises at least one therapeutic agent that may include at least one beta blocker. The at least one beta blocker may include, for example, without limitation, at least one of the following therapeutic agents: esmolol, propranolol, atenolol, oxprenolol, alprenolol, prindolol, timolol, acebutalol, metaprolol or nadolol.

In an embodiment, a method of delivering therapeutic agents comprises at least one therapeutic agent that may include at least one agent to treat cardiac arrhythmias. The at least one agent to treat cardiac arrhythmias may include, for example, without limitation, at least one of lidocaine, atropine or adenosine.

In an embodiment, a method of delivering therapeutic agents in vivo comprises at least one therapeutic agent to treat diabetes. These therapeutic agents include, for example, without limitation, glucagon or glucagon-like peptide-1.

In an embodiment, a method of delivering therapeutic agents comprises at least one therapeutic agent to treat neurological or behavioral disorders. The at least one agent to treat neurological or behavioral disorders include, for example, without limitation, venlafaxine.

In an embodiment, a method of delivering therapeutic agents comprises at least one therapeutic agent to treat animal airway passages, such as albuterol.

In an embodiment, a method of delivering therapeutic agents comprises at least one therapeutic agent that may include at least one antibiotic agent. The at least one antibiotic agent may include erythromycin or a derivative thereof.

In an embodiment, a method of delivering therapeutic agents comprises at least one therapeutic agent that may include at least one cholesterol-lowering therapeutic agent. In an embodiment, the at least one cholesterol-lowering therapeutic agent may include, for example, without limitation, atorvastatin.

In an embodiment, a method of delivering therapeutic agents comprises providing at least one of a catheter, a radioactive seed, a stent, a needle, a syringe, a trocar, a microchip or a tube. Furthermore, the method may include at least one reservoir, which may include, for example, without limitation, one of a lipid bilayer, a microcapsule, a liposome, a granule, capsule, a catheter, a radioactive seed, a stent, a needle or a tube.

An aspect of the disclosure includes a system. The system comprises a means for providing non-systemic delivery of at least one therapeutic agent to a local area of the animal in a therapeutically-effective concentration, wherein the therapeutically-effective concentration is in excess of a concentration that would produce a toxic effect if or when administered systemically to the animal, and wherein the at least one therapeutic agent has a short half-life. Furthermore, the system includes, without limitations, all the features and characteristics of an implantable device that was described above, and further includes an accompanying method of delivering at least one therapeutic agent to an animal, which have been described above in the summary and claimed below.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is an illustrative example of a bioavailability graph;

FIG. 2 is an illustrative example of a clinical response graph;

FIG. 3 is an illustrative example of a bodily clearance graph.

FIG. 4 is a schematic of an implantable medical device;

FIG. 5 is a schematic of an implantable medical device;

FIG. 6 is a schematic of an implantable medical device;

FIG. 7 is a schematic of an implantable medical device;

FIG. 8 is a schematic of human patient implanted with therapeutic agent delivery devices;

FIG. 9 shows an illustrative example of classes of therapeutic agents which may be used for non-systemic treatment;

FIG. 10 shows an illustrative example of classes of therapeutic agents which may be used for non-systemic treatment;

FIG. 11 shows an illustrative example of therapeutic agents;

FIG. 12 shows an illustrative example of therapeutic agents;

FIG. 13 shows an illustrative example of therapeutic agents;

FIG. 14 shows an illustrative example of therapeutic agents;

FIG. 15 shows an illustrative example of therapeutic agents;

FIG. 16 shows an illustrative example of therapeutic agents;

FIG. 17 shows an illustrative example of therapeutic agents; and

FIG. 18 shows an operational flow of a method of non-systemic delivery of therapeutic agents.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The following disclosure is drawn to medical devices, which may be implanted, that deliver to a localized site(s) in an animal body therapeutic agents that have, inter alia, short half-lives. In an embodiment, the short half-lives (or half-life) are no longer than approximately 0-5 hours. The half-life of a therapeutic agent described herein may be 0-4 hours, 0-3 hours, 0-2 hours or 0-1 hours. Further, such half-life may be from approximately 1-2 hours, 1-3 hours, 1-4 hours, 1-5 hours, 2-3 hours, 2-4 hours, 2-5 hours, 3-4 hours, 3-5 hours or 4-5 hours. As used herein, the term “half-life” (or “half-lives”) refers to the time it takes for the concentration of the therapeutic agent to be reduced by half of an initial concentration. Therapeutic agent half-lives may be affected by many factors such as, metabolic rates, therapeutic agent formulation chemistry, therapeutic agent solubility, serum albumin-therapeutic agent binding, protein-binding, hepatic first-pass metabolic pathways, renal elimination pathways, fecal excretion pathways, therapeutic agent volume distribution, degree of therapeutic agent ionization etc.

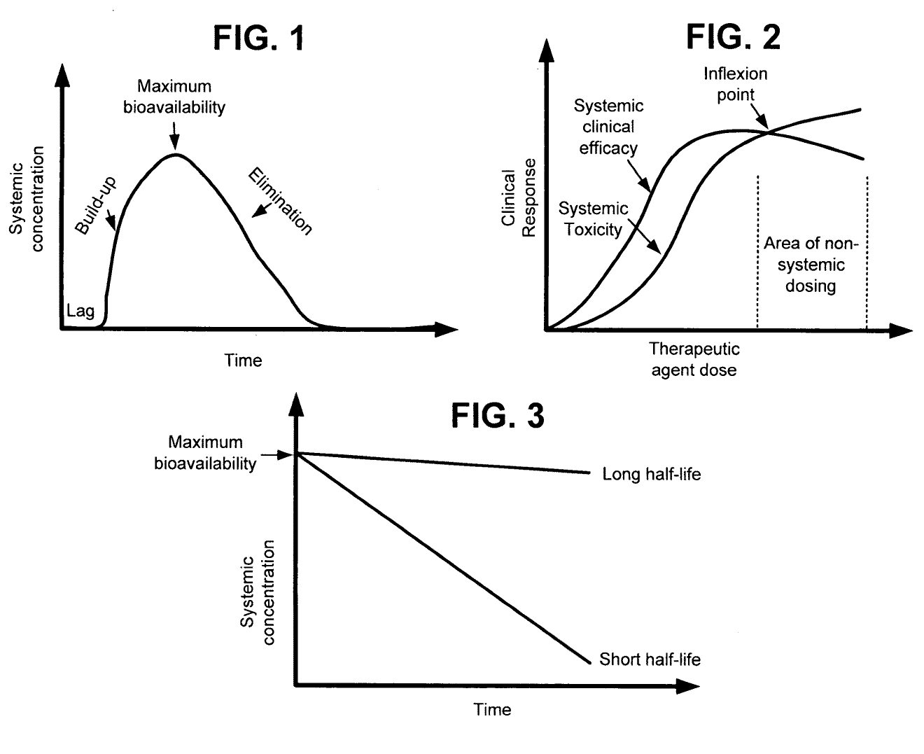

FIGS. 1-3 provide a conceptual framework for the content of the disclosure. FIG. 1 shows a graphical illustration of a bioavailability curve for a therapeutic agent (or a metabolite therefrom) that was orally administered to an animal in a single dose. Systemic concentration (meaning blood plasma concentration) of the therapeutic agent (or a metabolite therefrom) is plotted on the ordinate against time after ingestion on the abscissa. In a typical systemic circulation situation, the bioavailability (as measured in terms of the concentration of therapeutic agent) follows a sigmoidal relationship with time before maximum bioavailability. After an initial lag period, there may be a build-up phase wherein the systemic circulation concentration of the therapeutic agent increases with absorption time, reaching a maximum concentration for bioavailability, following which, the bioavailability declines. The time scale of the various phases depicted in FIG. 1 may vary based on a large number of factors that influence bioavailability of any given therapeutic agent. For example, systemic plasma concentration of a therapeutic agent may be influenced, among other things, by the mode of delivery (e.g., oral delivery vs. bolus injection or gradual intravenous injection) or therapeutic agent formulation. Likewise, different therapeutic agents exhibit different bioavailability indices based on, for instance, differing rates of elimination.

In an embodiment, the term “therapeutic agent(s)” includes, but is not limited to, chemical compounds, drugs, pharmaceuticals, medicines, medications, nutritionals, supplements, nutraceuticals, biomolecules, biological reagents, and animal or plant byproducts.

In an embodiment, the term “systemic concentration” mean the concentration of a therapeutic agent within the blood stream of an animal following systemic therapeutic agent administration.

In an embodiment, “bioavailability” means the fraction of an administered therapeutic agent dose that becomes available at the site of action.

The term “hepatic first pass” means terminal metabolism of an administered therapeutic agent by liver cells as the therapeutic agent passes through an animal's liver.

By “therapeutic agent volume distribution” it is meant the distribution and retention of a therapeutic agent in the various organs and tissues of an animal's body.

An embodiment is directed to treating non-systemically (or locally) an animal with at least one therapeutic agent that has a short half-life, wherein the at least one therapeutic agent is provided in a high local concentration that would normally cause toxicity if given systemically. However, as described below, if such doses are administered non-systemically at a localized site in an animal, because of the short-lived nature of the administered therapeutic agent, clinical benefit may accrue with limited systemic toxicity. FIG. 2 illustrates pharmacokinetic diagrams. Here, clinical response may be any type of therapeutic effect that may accrue to a patient. For example, clinical response may include, but is not limited to, pain relief or lowering of cholesterol. Clinical response may also include responses such as lack of, or reduced, toxicity. Looking at FIG. 2, at low doses of a therapeutic agent there may be very little, if any, clinical benefit. However, as the dose is increased clinical efficacy may increase. Higher doses of the therapeutic agent may produce a plateau in the beneficial clinical response, and in some instances there may even be a decrease in beneficial clinical response. If one were to measure a clinical response in terms of systemic toxicity, as illustrated in FIG. 2, systemic toxicity may be lower at therapeutic agent doses that may produce high clinical efficacy. On the other hand, if therapeutic agent doses are increased, systemic toxicity may reach and cross an imaginary inflexion point (or cross-over point) with systemic clinical efficacy. Farther up from the inflexion point dose, systemic toxicity may rise to higher levels than the clinical efficacy levels. The doses of a therapeutic agent that may be higher than the inflexion point dose are defined herein as falling within an “area of non-systemic dosing.” Doses falling within this area may be of utility in non-systemic therapy using short-lived therapeutic agents.

The spatial relationship between the systemic clinical efficacy curve and systemic toxicity curve may differ for different therapeutic agents. Furthermore, the inflexion point may occur at lower doses where systemic clinical efficacy may not have reached its peak. Other scenarios are also possible. For instance, for some therapeutic agents high systemic efficacy may be accompanied by low systemic toxicity even at high doses or high systemic efficacy may be accompanied by high systemic toxicity at low doses. Thus, clinical efficacy and clinical toxicity may be dose-dependent and dependent on the nature of the therapeutic agent. In relation to FIG. 2, at high systemic therapeutic agent doses for some therapeutic agents, the systemic clinical efficacy curve may closely parallel the toxicity curve. The result of these variations is that the inflexion or crossover point (see FIG. 2) may shift either to lower dose ranges or to higher dose ranges, depending on the spatial relationship between clinical efficacy curve and toxicity curve in clinical trials. Thus, the non-systemic dosage within the area (FIG. 2) may differ depending on the nature of the therapeutic agent used, the modes of administration, the formulation chemistry etc.

FIG. 3 illustrates the serum concentration of therapeutic agents over time. If two different therapeutic agents, one having a long half-life and another having a short half-life are present in an animal's body at maximum bioavailability, i.e., at the highest systemic concentration, then as shown in FIG. 3, the serum concentration of the therapeutic agent with a short half-life will decrease faster than the therapeutic agent with a long half-life. Thus, according to an embodiment, a therapeutic agent with an extremely short half-life may be potentially used at a higher-than-systemic concentration at a localized area of an animal body, with minimal serious adverse events, because of rate of decrease in concentration of the short-lived therapeutic agent is relatively rapid. Therefore the therapeutically-effective concentration of a short-lived therapeutic agent could be in excess of a concentration that would otherwise produce a toxic effect when administered systemically (FIG. 2).

As used herein, the terms “toxicity” or “toxic effect” refer to any serious adverse event such as an untoward medical occurrence that may present itself during treatment or during administration with a therapeutic agent. The event may include, but is not limited to death or a life-threatening event; or requires inpatient hospitalization or prolongation of existing hospitalization; or creates persistent or significant disability/incapacity, or a congenital anomaly/birth defects. See Title 21 C.F.R. Sec. 312.32, which is incorporated herein by reference.

As used herein, the term “therapeutically-effective” concentration includes, among other things, a concentration of a therapeutic agent that causes a beneficial effect for an animal. Beneficial effects includes, modulating, controlling, maintaining or reducing a diseased state or any other state. Additionally or alternatively, beneficial effects include any positive effects produced as a result of treating an animal patient (including humans) with a therapeutic agent.

A number of publications, for example, “Basic Clinical Pharmacology” by Bertram G. Katzung (Lange Medical Books/McGraw-Hill Medical, New York; 8th edition, 2001), which is incorporated herein by reference, discusses the basic principles of pharmacology.

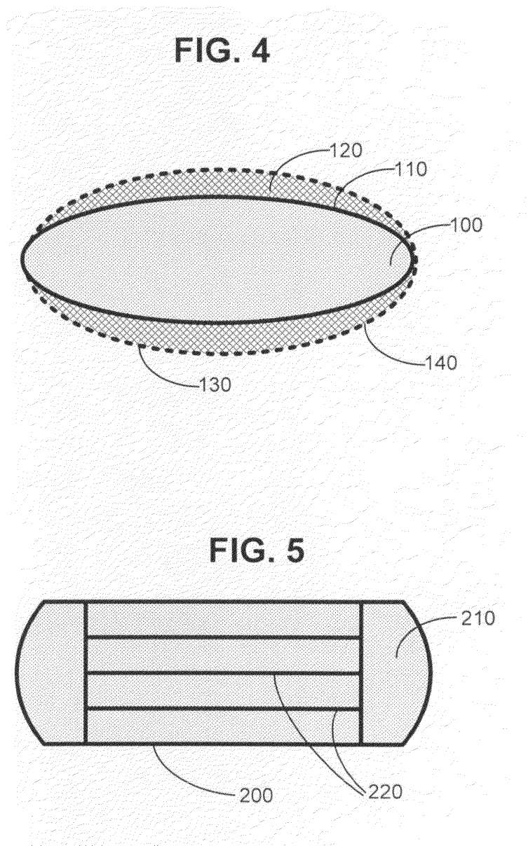

In an aspect, an implantable medical device is described herein comprising at least one reservoir holds at least one therapeutic agent. In an embodiment, the at least one reservoir includes one of a lipid bilayer, a microcapsule, a liposome, a granule, a capsule, a catheter, a radioactive seed, a stent, a needle or a tube. For example, in FIG. 4, there is illustrated a schematic of a microcapsule or a liposome, which may hold a therapeutic agent. The therapeutic agent 100 may be held within a sac-like structure 110, which may be made from polymer such as a hydrophobic polymer layer. In an embodiment, the outer shell of the microcapsule may comprise multiple membrane layers 120, 130, which may be alternatively made from hydrophilic or hydrophobic polymer agents. In another embodiment the outermost layer may comprise porous semi-permeable membrane 140. These types of therapeutic agent-reservoirs are well-known in the field. For instance, U.S. Pat. Nos. 3,565,559, 5,332,584, 6,099,864, 5,384,133, 5,674,519 and 6,534,091, which are incorporated herein by reference, disclose processes for making microcapsules used for therapeutic agent delivery.

FIG. 5 schematically illustrates an embodiment of a reservoir that may deliver therapeutic agents. The reservoir may be a tube-like implantable device 200 filled with a therapeutic agent 210. In some embodiments, the tube-like implantable device may have multiple chambers 220 for holding more than one therapeutic agent.

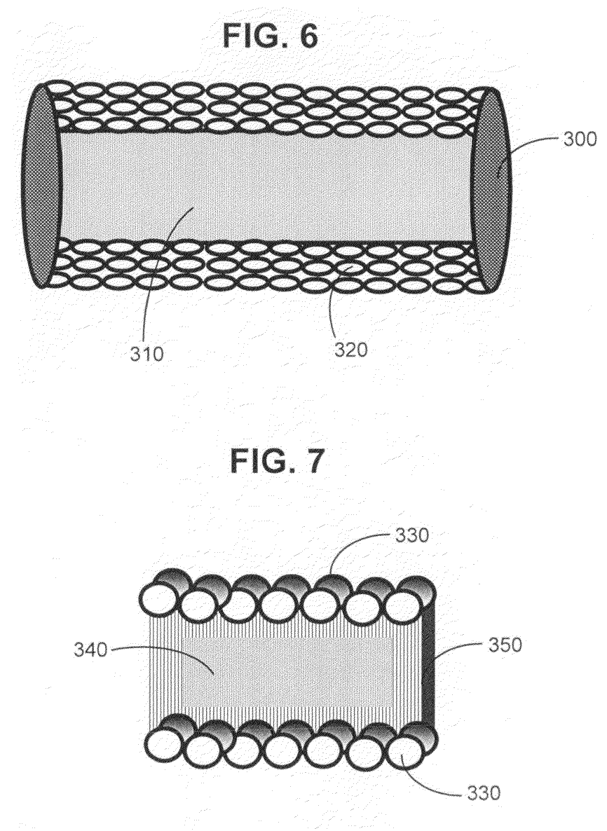

There is presented in FIG. 6, an embodiment of an implantable device that is a stent 300, which may serve as a reservoir for holding a therapeutic agent 310. The stent-like body may comprise a wire mesh 320 (or any other type of mesh) and may be used to keep a blood vessel open and for therapeutic agent diffusion to the outside. In an embodiment, a therapeutic agent may comprise an elution surface, which may release a therapeutic agent or prodrug by diffusion. Drug-eluting stents have been described in a number of U.S. patents, for example U.S. Pat. Nos. 5,591,227, 5,599,352 and 6,702,850, which are incorporated herein by reference.

In FIG. 7, there is illustrated a schematic for an embodiment of an implantable therapeutic agent-holding reservoir such as a liposome, which may include a lipid bilayer. In an embodiment, the lipid-bilayer may comprise, for example, hydrophilic phosphate-containing heads 330. A therapeutic agent 340 may be held in a medium comprising, for example, hydrophobic tails 350. U.S. Pat. Nos. 5,981,501, 5,922,594 and 5,718,914, which are incorporated herein by reference, disclose methods of making and using lipid bilayers.

A number of U.S. patents describe the use of various other types of implantable reservoirs and methods thereof for delivery of therapeutic agents. For example, U.S. Pat. Nos. 5,667,798, 6,576,257, 6,911,455, 6,099,864, 6,727,203, 7,053,134, 7,066,873, and WIPO publication number WO0023052, which are incorporated herein by reference, disclose therapeutic agent-delivery devices.

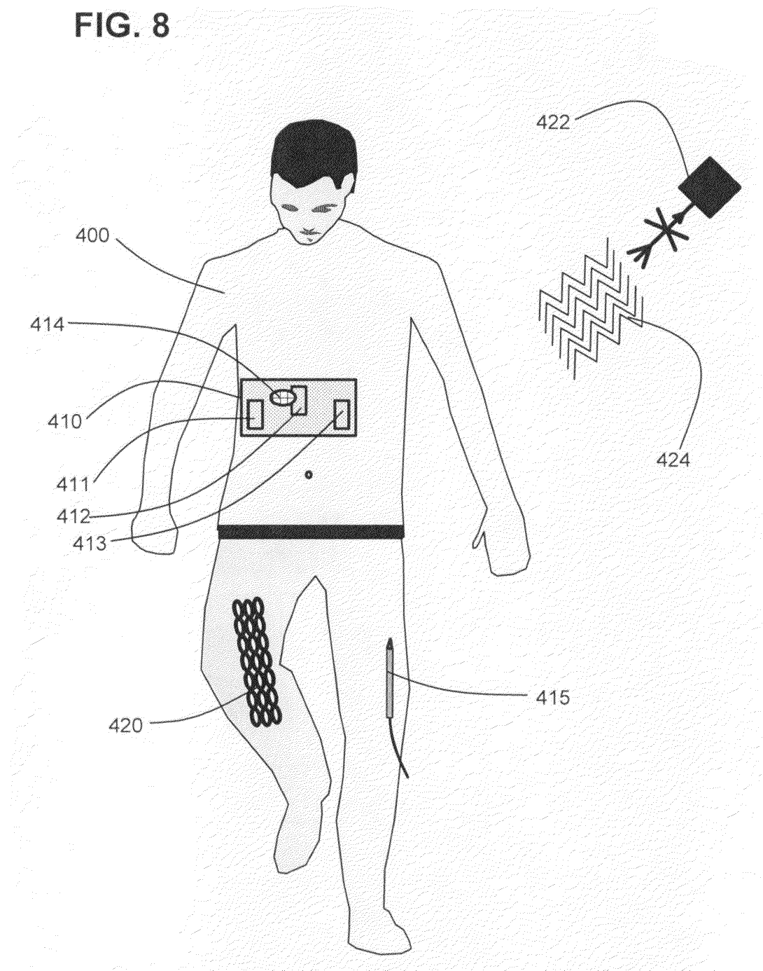

An embodiment includes non-systemic delivery of at least one therapeutic agent having short-half life to at least one local area of an animal in a therapeutically-effective concentration. FIG. 8 depicts examples of therapeutic agent delivery devices implanted within a human patient 400. The devices may be implanted temporarily or permanently. For example, the devices may be located in the abdomen area 410, knee joint area 420 or thigh area 415. The implanted device 410 may have multiple components 411, 412, 413, each performing a different function such as delivering a different therapeutic agent. In an embodiment, the multi-component device may comprise subcomponents that may function in concert. In an embodiment, a delivery device may provide a very high local concentration that is not toxic. However, if the therapeutic agent reaches the blood stream it is degraded, metabolized, eliminated, protein-bound, or otherwise decreases in concentration in the blood such that the likelihood of a toxic effect is significantly reduced. In another embodiment, the delivery device may include a catheter 415 or a stent or a stent-like device 420.

As used herein, “therapeutic agent delivery device” means a device that is configured to deliver or delivers a form of a therapeutic agent to a local area within a patient. The therapeutic agent may be in any form such as a solid, a fluid or a therapeutic agent that is fluidizable, or a gas. In an embodiment, the delivery device may be capable of delivering a therapeutic agent to a local area in human body. For example, a therapeutic agent may comprise a compound that exhibits a physiological effect, a pharmaceutical therapeutic agent or its pharmaceutically acceptable salt, metabolite, adduct or derivative, a biological, a chemical compound, a peptide or nucleotide or glycopeptide or lipopeptide, a nutrient or micronutrient, a vitamin, a nutraceutical therapeutic agent, or any combination thereof. A therapeutic agent may be a biologically active therapeutic agent, including a cell, cell component, virus, provirus, or microscopic lifeform. In some embodiments, a therapeutic agent may include at least one nutrient, hormone, growth factor, medication, chemical compound, enzyme, genetic therapeutic agent, vaccine, vitamin, neurotransmitter, cytokine, cell-signaling, pro- or anti-apoptotic agent, imaging agent, labeling agent, diagnostic compound, nanotherapeutic agonist agent, inhibitor, antagonist or blocker. In some embodiments, the therapeutic agent may be a component or precursor of a biologically active therapeutic agent; for example, the therapeutic agent may include at least one precursor or component of a nutrient, hormone, growth factor, medication, therapeutic compound, enzyme, genetic therapeutic agent, vaccine, vitamin, neurotransmitter, cytokine, cell-signaling therapeutic agent, pro- or anti-apoptotic agent, imaging agent, labeling agent, diagnostic compound, nanotherapeutic agent, inhibitor, or blocker. Such precursors, may include, for example, prodrugs (see, e.g., “Liver-Targeted Drug Delivery Using HepDirect1 Prodrugs,” Erion et al., Journal of Pharmacology and Experimental Therapeutics Fast Forward, JPET 312:554-560, 2005 (stating a first publication date of Aug. 31, 2004) and “LEAPT: Lectin-directed enzyme-activated prodrug therapy”, Robinson et al., PNAS Oct. 5, 2004 vol. 101, No. 40, 14527-14532, stating published online before print Sep. 24, 2004, both of which are incorporated herein by reference. Beneficial therapeutic agents may be produced, for example, by conversion of prodrug to drug by enzymatic reaction of therapeutic agent in the bloodstream or a tissue or an organ (CYP450, cholesterol metabolism, e.g., with cholesterol monooxygenase, cholesterol reductase, cholesterol oxidase). Some commercial entities have been manufacturing instruments and tools for targeted therapeutic agent delivery. For example, Durect Corporation is a manufacturer of catheters, direct injection equipment etc.

Returning to FIG. 8, a delivery device described herein may be used to controllably deliver therapeutic agents aimed at the prevention or treatment of diseases. Embodiments of a therapeutic agent delivery device may optionally include, for instance, a remotely activatable or a signaling device 422 that may control or regulate at least one therapeutic agent delivery device. In an embodiment, the activatable or signaling device 422 may emit a control signal 424 that may communicate information or data to an implanted device 410. The control signal 424 may be provided, for example, either prior to deployment in vivo of a therapeutic agent delivery device, or subsequent to deployment in vivo. The control signal may include steps for a high concentration, short duration therapeutic regime, or steps for a long-term delivery regime of at least one therapeutic agent. In the embodiment of FIG. 8, therapeutic agent delivery device 410 may include a controllable output mechanism 414 and reservoir 412, which may contain therapeutic agents. In an embodiment, signaling device 422 may transmit a control signal 424 to the responsive controllable output mechanism 414 to control the exit of a therapeutic agent from the reservoir 412. In an embodiment, a therapeutic agent may exit from the reservoir 412 in a regulated manner according to a programmed dose regime. Therapeutic agent delivery regimen may include, for example, daily delivery of at least one therapeutic agent for a period of days, or weeks or months. Delivery may be scheduled in a constant manner to permit consistent and maintained localized levels of a therapeutic agent. Some delivery regimen may take into account the pharmacokinetic properties of the therapeutic agent in order to maintain a desired concentration of the therapeutic agent. As described herein, some embodiments of the therapeutic agent delivery device may include sensors that are configured to sense or senses a biological condition or other parameter of a patient's environment. The therapeutic agent delivery device may be programmed to respond to a sensed condition or parameter. Some diseases or infections may require delivery of at least one therapeutic agent over the course of months in order to prevent a recurrence of a disease. The therapeutic agent delivery devices described herein allows for myriad variations in delivering therapeutic agents to an animal, and in particular a human. Any aspect of the function of the therapeutic agent delivery device including, but not limited to, the timing and quantity or dose of therapeutic agent delivery, may be programmed into the delivery device or may be controlled by a remote controller, as desired by a user.

In an embodiment, the following list of disease states or biological conditions, without limitation, may be amenable to localized therapy with therapeutic agents with short half-lives:
1. Tumors: Non-systemic delivery of high doses of therapeutic agents to tumors, including, but not limited to sarcomas of the limbs and brain tumors.
2. Blood vessel malformations: Non-systemic delivery of high doses of therapeutic agents to, for example, arteriovenous malformation in the brain, which may require sclerosis. Other examples may include, but not limited to hemorrhoids and varicose veins.
3. Single organ diseases: Non-systemic delivery of high doses of therapeutic agents to, for example, the penis in cases of erectile dysfunction. Localized delivery of a vasodilator to the penis could avoid problematic cardiac and peripheral vascular problems associated with systemic delivery of therapeutic agents.
4. Pain relief: Non-systemic high doses of therapeutic agents may be used to locally deliver narcotic or other pain relieving substances to a painful area of a body without systemic side-effects (e.g. constipation) or possibly without addictive side-effects.
5. Local infections: Non-systemic delivery of high doses of antibiotics to infected sites without need for systemic toxicities.
6. Bone growth: Non-systemic delivery of high doses of therapeutic agents to bone or portions thereof. This treatment could potentially provide bone growth enhancing factors (e.g. bone morphogenic protein) to enhance bone healing or fusion of an orthopedic construct.
7. Cardiac or brain recovery from heart attack/stroke: Localized delivery of angiogenic short-lived therapeutic agents to promote revascularization of injured or hypoxic tissue may be a treatment procedure for these conditions.
8. Body modeling/plastic surgery modifications: Localized delivery of therapeutic hormone agents to breast tissue or other parts of the anatomy, which may have undergone surgery, could be targeted for short-lived therapeutic agent delivery for healing/treatment purposes.

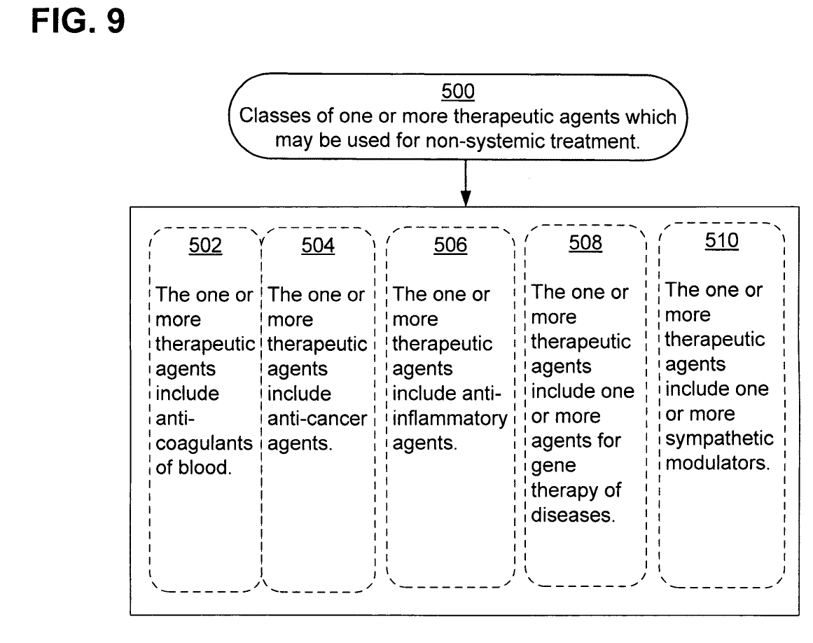

There are shown in FIG. 9, examples of classes of at least one therapeutic agent, which may be used for non-systemic treatment 500. In an embodiment, the at least one therapeutic agent may include anti-coagulants of blood 502. In another embodiment, the at least one therapeutic agent includes anti-cancer agents 504. Additionally, the at least one therapeutic agent includes anti-inflammatory agent 506. In an embodiment, the at least one therapeutic agent includes at least one agent for gene therapy of diseases 508. In another embodiment, the at least one therapeutic agent includes at least one beta blocker 510.

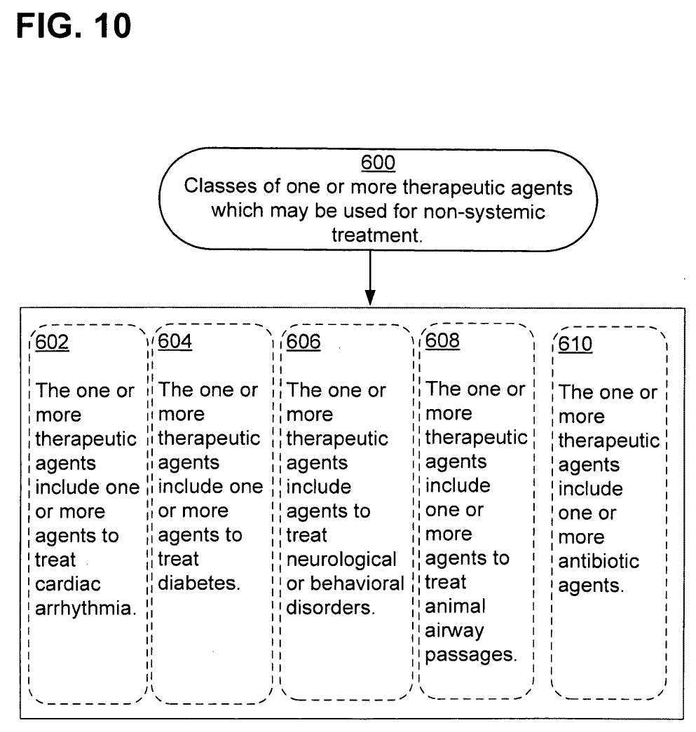

Additionally or alternatively, as shown in FIG. 10, in an embodiment, classes of at least one therapeutic agent which may be used for non-systemic treatment 600 may include the following: at least one therapeutic agent includes at least one agent to treat cardiac arrhythmia 602. In yet another embodiment, the at least one therapeutic agent includes at least one agent to treat diabetes 604. In another embodiment, the at least one therapeutic agent includes at least one agent to treat neurological or behavioral disorders 606. Furthermore, the at least one therapeutic agent includes at least one agent to treat animal airway passages 608. In another embodiment, the at least one therapeutic agent includes at least one antibiotic agent 610 to treat bacterial or microbial infections.

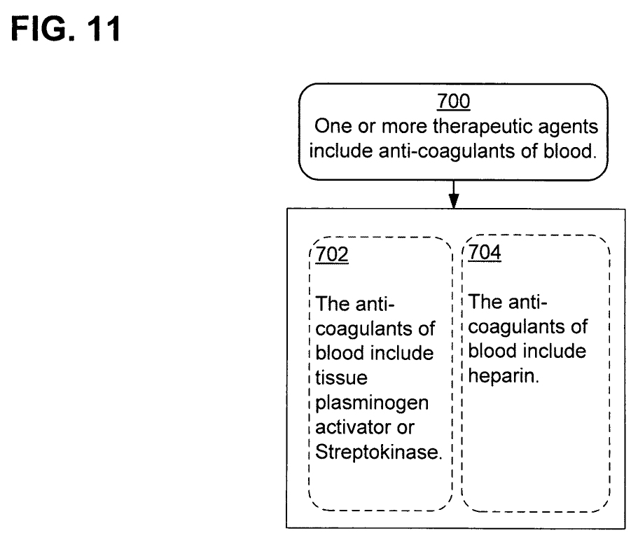

In an embodiment, at least one therapeutic agent includes an anti-coagulant of blood (see FIG. 11, item 700). Examples of anti-coagulants of blood with short half-lives include tissue plasminogen activator (TPA), streptokinase 702 or heparin 704.

TPA has been used as a thrombolytic therapeutic agent for acute myocardial infarction. TPA has a circulation half-life of approximately 5 minutes. TPA is manufactured by Genentech, Inc. of South San Francisco. For example, U.S. Pat. Nos. 4,853,330, 5,106,741, 5,658,788, 5,648,250, which are incorporated herein by reference, disclose the synthesis and use of TPA and its derivatives.

Similar to TPA, streptokinase is another thrombolytic agent used to treat cardiac disease. Streptokinase has a half-life of about 20 minutes. For example, U.S. Pat. Nos. 3,980,772, 3,639,213, 4,808,405 and 3,226,304, which are incorporated herein by reference, disclose the synthesis and use of streptokinase and its derivatives.

Heparin is another blood-clot dissolving substance, with a half-life of around 1.5 hours. For instance, U.S. Pat. Nos. 4,703,042, 5,280,016 and 5,039,529, which are incorporated herein by reference, disclose the synthesis and use of heparin and its derivatives.

In an embodiment, the above-mentioned therapeutic agents may be used at therapeutically-effective concentrations in excess of concentrations that would otherwise produce a toxic effect when administered systemically to a human. In certain embodiments these therapeutic agents could be used in localized areas such as specific arteries.

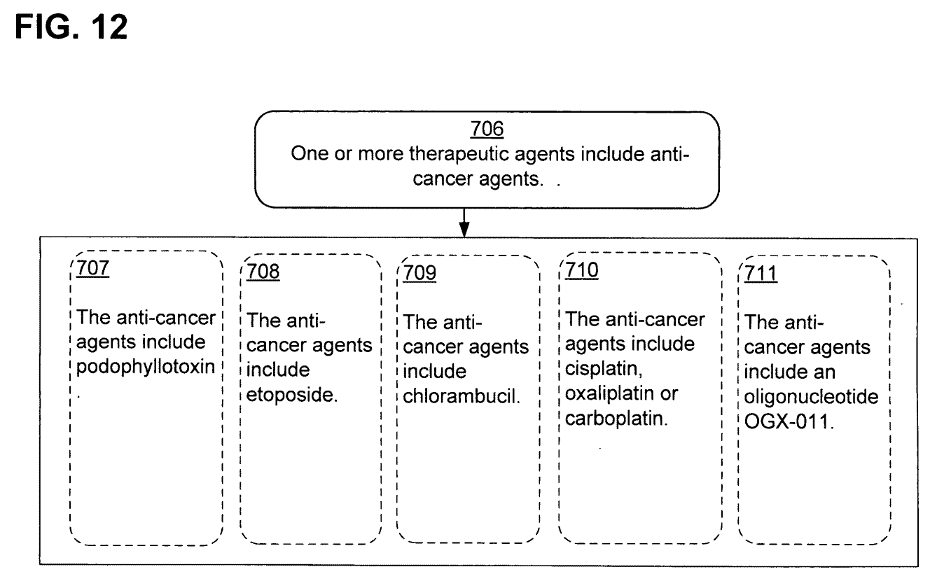

In an embodiment, as illustrated in FIG. 12, at least one therapeutic agent that may be used in non-systemic therapy includes anti-cancer agents 706.

In an embodiment, the anti-cancer agents include podophyllotoxin 707. The half-life of podophyllotoxin has been reported to be about 1.0 to 4.5 hours. For instance, U.S. Pat. Nos. 4,680,399, 4,567,253, 4,900,814 and 5,057,616, which are incorporated herein by reference, disclose preparation and use of podophyllotoxin.

Another anti-cancer agent is etoposide 708 (FIG. 12), which has a half-life of about 3 hours in children. For example, U.S. Pat. Nos. 4,757,138, 4,713,246, 4,701,327 and 6,872,841, which are incorporated herein by reference, disclose preparation and uses of etoposide.

In an embodiment, anti-cancer agent includes chlorambucil 709. Chlorambucil has a half-life of about 1.5 hours in human. For instance, U.S. Pat. Nos. 4,332,797, 4,835,182, 4,938,897 and 5,602,278, which are incorporated herein by reference, disclose preparation and use of chlorambucil.

Another embodiment includes the anti-cancer agents: cisplatin, oxaliplatin or carboplatin 710. The non-protein bound platinum derivatives have an estimated half-life of about 1.3 hours. For instance, the initial phase of elimination half-life for cisplatin is 8 to 49 minutes in normal patients. Examples of U.S. patents that disclose preparation and use of cisplatin include U.S. Pat. Nos. 4,310,515, 5,945,122, 4,645,661 and 5,922,689, which are incorporated herein by reference.

For oxaliplatin, the half-life is approximately 10-25 minutes. For carboplatin the estimated half-life is about 1-2 hours. For instance, U.S. Pat. Nos. 6,306,902, 6,673,805, 7,208,616 and 6,602,870, which are incorporated herein by reference, disclose methods of preparation and clinical uses of oxaliplatin. Compositions and clinical uses of carboplatin have been disclosed, for example, in U.S. Pat. Nos. 5,104,896, 5,620,703, 6,548,541 and 6,037,336, which are incorporated herein by reference.

In systemic therapy for colon cancer, oxaliplatin is reportedly used at 85 mg per meter-squared i.e., 145 mg total dose for an average person weighing 65 kg. Assuming that an average person has 5 liters of blood, the blood plasma concentration is approximately 3 micrograms per milliliter, assuming even distribution and constant rate of elimination. In an embodiment, a concentration greater than 85 mgs could be delivered to a local area for treatment with reduced toxicities.

An embodiment of a therapeutic agent for cancer therapy includes OGX-011 (see FIG. 12, item 711), a 2′-methoxyethyl antisense oligonucleotide, having a plasma half-life of approximately 2-3 hours. Use of this compound as a potential therapeutic agent for prostate cancer treatment was discussed in Journal of the National Cancer Institute Vol. 97, pp 1287-1296 (2005), which is incorporated herein by reference. Additionally, for example U.S. Pat. Nos. 5,582,986, 5,801,154, 5,242,906 and 5,576,208 disclose the preparation and uses of antisense oligonucleotides for cancer therapy. Briefly, antisense oligonucleotides are DNA-based or RNA-based reagents that prevent mRNA translation by reversibly binding to target mRNA sequences. Alternatively, antisense oligonucleotides may be directed to block gene transcription by binding to target DNA.

In an embodiment, it is predicted that the above-mentioned therapeutic agents may be useful for localized treatment of certain tumors at higher than systemic concentrations.

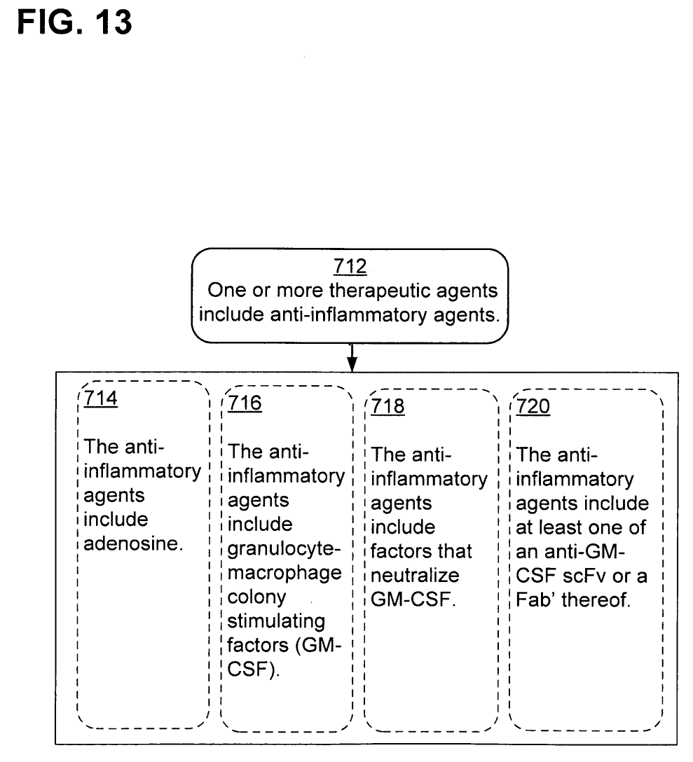

As illustrated in FIG. 13, in an embodiment, at least one therapeutic agent includes anti-inflammatory agents 712. Anti-inflammatory agents include, but are not limited to adenosine 714, granulocyte-macrophage colony stimulating factors (GM-CSF) 716, factors that neutralize GM-CSF 718 and at least one of an anti-GM-CSF scFv or a Fab′ thereof 720.

Adenosine is a potent anti-inflammatory agent and a cardiac therapeutic agent with an extremely short half-life (about a few seconds). In an embodiment, adenosine is used systemically at 12 mg for maximum dose in treating cardiac arrhythmia. In an embodiment, the method described herein includes administering adenosine at greater 12 mg upstream at an atrioventricular node in the heart, as local non-systemic therapeutic agent.

For example, U.S. Pat. Nos. 4,673,563, 5,106,837, 5,244,896, 6,955,814 and 6,221,851, which are incorporated herein by reference, provide disclosures directed to preparation and therapeutic applications of adenosine.

Granulocyte-macrophage colony stimulating factors (GM-CSF) and antibodies that neutralize GM-CSF have been developed as anti-inflammatory therapeutic agents (see for example, Clinical Cancer Research vol. 5, pp 1353-1361; New England Journal of Medicine Vol. 332, pp 1671-1677, which are incorporated herein by reference), provides disclosures directed to therapeutic applications of GM-CSF. Sargramostim is the only GM-CSF with Food and Drug Administration (FDA)-approved labeling. Sargramostim was approved to accelerate myeloid recovery after autologous bone marrow transplantation and for use in patients with delayed or failed engraftment after allogeneic or autologous bone marrow transplantation. Normally CSFs are used systemically. IV infusion of 20 mcg/kg of NEUPOGEN® (Filgrastim) over 24 hours may result in mean and median serum concentrations of approximately 48 and 56 ng/mL, respectively. Subcutaneous administration of 3.45 mcg/kg and 11.5 mcg/kg may result in maximum serum concentrations of 4 and 49 ng/mL, respectively, within 2 to 8 hours. It is surmised that higher than systemic doses of NEUPOGEN® (Filgrastim) may be used for local application in cancer patients because the half-life of NEUPOGEN® (Filgrastim) is about 3.5 hours.

For example, U.S. Pat. Nos. 5,032,395, 5,073,627, 5,679,356 and 5,942,253, which are incorporated herein by reference, disclose methods for preparation of GM-CSF and methods of therapeutic application of the same.

The methods of preparation of whole immunoglobulin (IgG) from animals such as mouse (or rabbit, rat, guinea pig, goat etc) and Fab′ fragments therefrom are well-established in the art. A number of well-characterized antibody targets have been commercially exploited. Some currently commercial antibody therapy targets include proteins such as, HER-2, CD20, TNF, VEGF as well as CD22 and CD33.

A number patents disclose antibody methodology. For example, U.S. Pat. Nos. 4,814,433, 4,814,433 and 4,937,183, which are incorporated by reference disclose methods of preparing Fab′ fragments from intact IgG. Other publications such as (Ullman, et al, Methods in Enzymology, Vol. 74, p 28 (1981); Inoue, et al., Analytical Letters, vol. 18, p. 1331 (1985); and German Patent Application No. DE 3430905) and as immunotherapeutic agents (Smith, et al., Antibodies in Human Diagnosis and Therapy, Haber and Krause (Eds.), Raven Press, New York, N.Y., p. 365 (1977)) describe detailed procedures for antibody fragment preparations. These publications are incorporated herein by reference. Briefly, Fab′ are usually monovalent (or divalent) proteolytic fragments of whole intact IgGs. The advantage of using Fab′ fragments, as opposed whole IgGs, is that there is decreased interference from non-specific binding molecules and anti-species antibodies. The Fab′ fragments are further purified free of proteases before use. For example, human anti-GM-CSF scFv, which was experimentally tested as an anti-inflammation agent, was reported to have a half-life of about 2 hours (See Protein Engineering and Selection Vol. 19, pp 461-470), thus providing an opportunity for its localized therapeutic clinical application.

At least one of the above-mentioned therapeutic agents may be used for localized non-systemic treatment of areas in an animal at significantly higher than systemic doses because of the predicted short-half lives of the therapeutic agents.

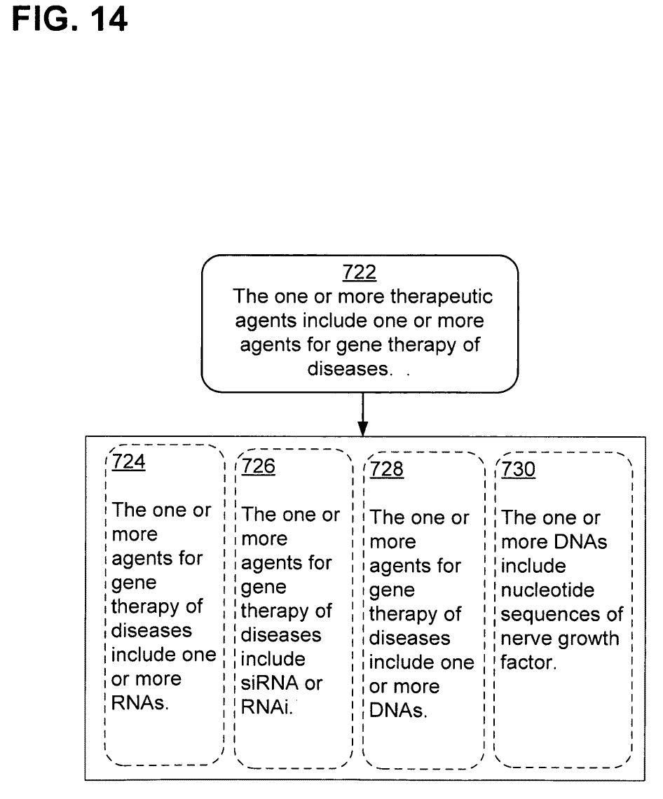

In an embodiment, at least one therapeutic agent includes at least one agent for gene therapy of diseases 722 (See FIG. 14). The term “disease” as used herein means, inter alia, a disease in which there is an abnormality in or a deficiency of a particular molecule, usually a DNA or RNA or a protein. The term “gene therapy” as used herein includes therapeutic techniques wherein a foreign DNA or RNA molecule is introduced into the body of a patient to complement a function of a gene deficiency or to silence the expression of a gene. In an embodiment, the at least one agent for gene therapy of diseases include at least one RNAs 724 such as siRNA 726 or RNAi 726 or DNAs 728. RNAi and siRNA molecules are examples of pre-translational (or post-translational after turnover) RNA interference technology used for gene silencing. RNA interference is a natural cellular mechanism by which RNA is recognized as “foreign” due to its existence in a double-stranded form. This results in the degradation of the double-stranded RNA, along with single-stranded RNA having the same sequence. Small interfering RNA (siRNA) guides sequence-specific degradation of the homologous mRNA, thus producing “knock-down” cells. Similarly, RNAi is a well-known gene silencer that is being developed as a potential therapeutic agent. siRNA and RNAi technology is well-established. RNAi molecules are designed in a gene-specific manner usually according to various known algorithms, for example, Tuschl's rules, which are incorporated herein by reference:

### General Guidelines

1. siRNA targeted sequence is usually 21 nt in length.
2. Avoid regions within 50-100 bp of the start codon and the termination codon.
3. Avoid intron regions.
4. Avoid stretches of 4 or more bases such as AAAA, CCCC.
5. Avoid regions with GC content <30% or >60%.
6. Avoid repeats and low complex sequences.
7. Avoid single nucleotide polymorphism (SNP) sites.
8. Perform BLAST homology search to avoid off-target effects on other genes or sequences.
9. Always design negative controls by scrambling targeted siRNA sequence. The control RNA should have the same length and nucleotide composition as the siRNA but have at least 4-5 bases mismatched to the siRNA. Make sure the scrambling will not create new homology to other genes.

### Tom Tuschl's Rules

1. Select targeted region from a given cDNA sequence beginning 50-100 nt downstream of start codon.
2. First search for 23-nt sequence motif AA (N₁₉). If no suitable sequence is found, then,
3. Search for 23-nt sequence motif NA(N₂₁) and convert the 3′ end of the sense siRNA to TT.
4. Or search for NAR(N₁₇)YNN.
5. Target sequence should have a GC content of around 50%.

### A=Adenine; T=Thymine; R=Adenine or Guanine (Purines); Y=Thymine or Cytosine (Pyrimidines); N=Any.

A number of companies offer custom-made RNAi molecules. For example, companies such as Alnylam Pharmaceuticals, Benetec and Dharmacon offer custom RNAi synthesis. In an embodiment, RNAi molecules may be delivered to a cell to destroy mRNA that is defective or diseased. Naked RNAi, delivered by injection, for instance, to an animal body may have a very short half-life (about a few minutes) because it is attacked by a plethora of serum RNases. However, chemically modified RNAi may have a longer half-life (about a few hours). In an embodiment, the delivery mechanism could be by way of an injection of naked, or liposome-encapsulated or microparticle-captured chemically and specifically-modified RNAi to a localized site in an animal body. For example, U.S. Pat. Nos. 6,712,617, 5,475,096, 7,175,999 and 7,208,154, which are incorporated herein by reference, disclose methods of preparation and use of RNA molecules as potential therapeutic agents.

In an embodiment, gene therapy may be designed to introduce genetic therapeutic agents into cells to compensate for abnormal genes or to make a beneficial protein, as for example sequences (not claimed herein) of nerve growth factor (NGF) (See FIG. 14, item 730). The short serum half-life of NGF (about 7.2 minutes as measured in the rat; (Brain Res. Mol. Brain Res. 36:280-286, 1996) may make possible direct localized use of NGF treatment at high dosages. In an embodiment, nerve growth factor could be potentially useful in the treatment of stroke-induced paralysis or Parkinson's disease.

For example, viral-mediated gene transfer to treat peripheral neuropathy in diabetic mouse models has been investigated (See Diabetes, Vol. 51, pp 2227-2232, 2002). U.S. Pat. Nos. 5,399,346, 5,240,846, 5,792,453 and 5,252,479, which are incorporated herein by reference, disclose vectors and methods for potential treatment of diseases.

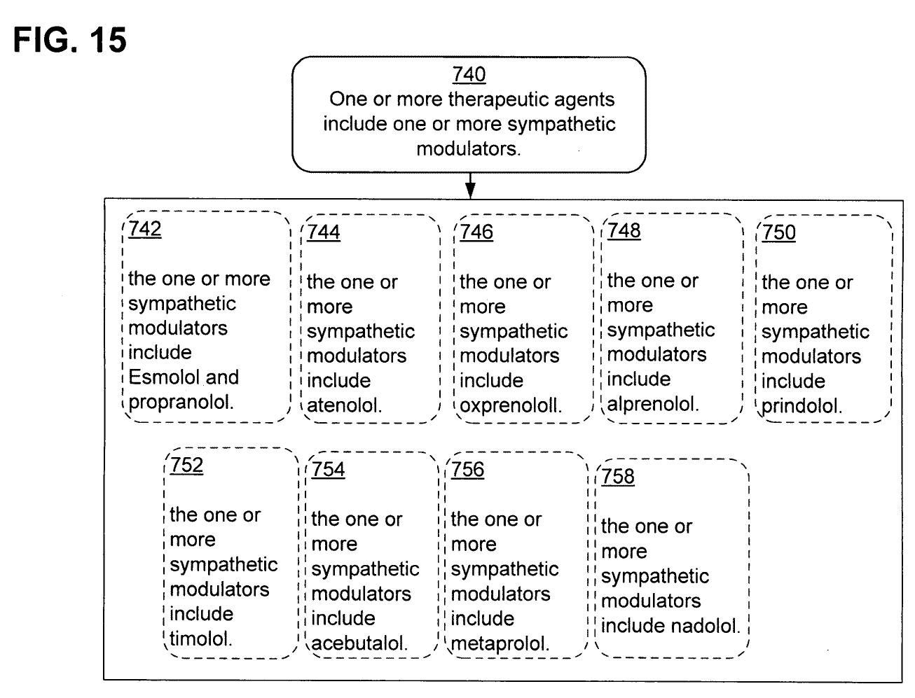

In an embodiment, at least one therapeutic agent includes sympathetic modulators such as beta blockers and alpha agonists. The at least one sympathetic modulators include beta blockers, also known as beta andrenergic blocking agents (See FIG. 15, item 740). Examples of beta blockers include esmolol and propranolol 742; atenolol 744; oxprenololl 746; alprenolol 748; prindolol 750; timolol 752; acebutalol 754; metaprolol 756; nadolol 758. Beta blockers have reportedly been used in the treatment of a variety of conditions, such as high blood pressure, glaucoma, cardiac arrhythmias and migraines. These agents in general have half-lives in the range of about 2 to 5 hours. Beta-blockers have been reported to be somewhat toxic because of their sodium and calcium channel-blocking properties.

For example, U.S. Pat. Nos. 4,178,374, 4,428,883, 4,794,111 and 4,966,914, which are incorporated herein by reference, disclose methods of preparation and uses of beta blockers for therapeutic treatment.

In an embodiment, it is suggested that sympathetic modulators such as beta blockers may be useful for the localized treatment of ocular diseases such as glaucoma or migraine at higher than systemic doses.

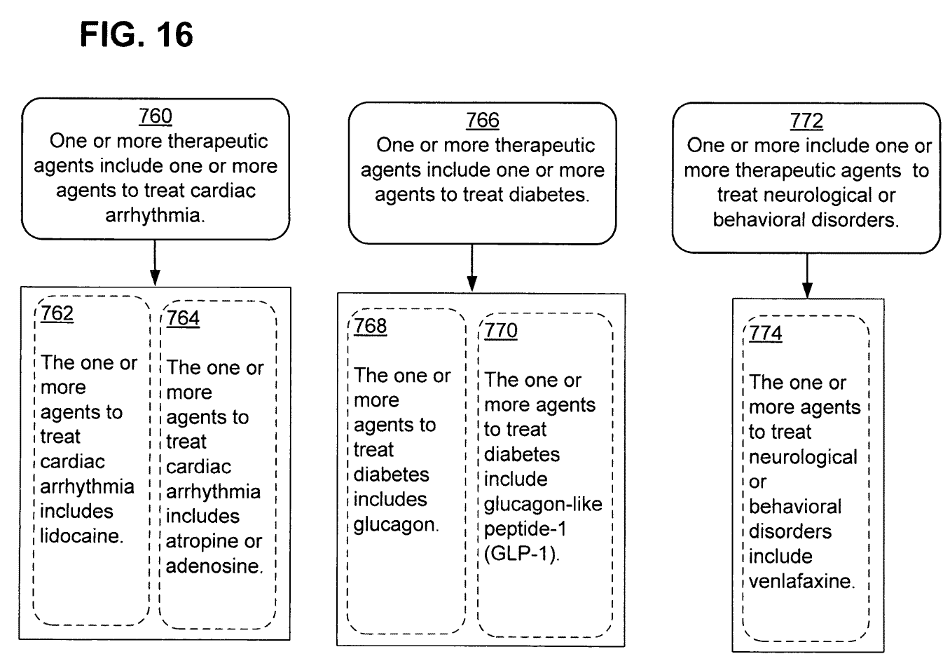

As shown in FIG. 16, in an embodiment at least one therapeutic agent includes at least one agent to treat cardiac arrhythmia 760. Examples of therapeutic agents that may be used in non-systemic therapy may include lidocaine 762 and atropine or adenosine 764. Lidocaine has a reported half-life of about 1.5-2 hours. Lidocaine has been reported to have potential uses as a short-term anesthetic in dentistry. Atropine also has a reported short half-life: about 2 hours. Adenosine is a potent cardiac therapeutic agent with an extremely short half-life (about a few seconds). In an embodiment, adenosine is used systemically at 12 mg for maximum dose in treating cardiac arrhythmia. In an embodiment, adenosine may be administered upstream at an atrioventricular node in the heart, as local non-systemic therapeutic agent.

For example, U.S. Pat. Nos. 4,673,563, 5,106,837, 5,244,896, 6,955,814 and 6,221,851, which are incorporated herein by reference provide disclosures directed to preparation and therapeutic applications of adenosine.

Numerous U.S. patents have disclosed the clinical applications of lidocaine and atropine as therapeutic agents. For example, U.S. Pat. Nos. 3,080,327, 4,659,714, 4,406,883 and, which are incorporated herein by reference disclose pharmaceutical compositions and uses of lidocaine. Likewise, U.S. Pat. Nos. 3,450,814, 3,901,967, 3,520,975, 4,952,586, and 5,612,027, which are incorporated herein by reference, provide disclosure of methods of use and compositions of atropine and its derivatives.

In an embodiment, direct local treatment to portions of a patient's heart with atropine may be a potential therapeutic strategy.

In an embodiment, at least one therapeutic agent includes at least one agent to treat diabetes (FIG. 16, item 766). Examples of therapeutic agents with short half-lives for treatment of diabetes include glucagon 768 and glucagon-like peptide-1 (GLP-1) 770.

Glucagon and GLP-1 have been reportedly used for treatment of diabetes and hypoglycemia. Typically, the half-lives of glucagon or GLP have been reported to be about 1.5-5 minutes (Eur. J. Endocrinol. vol. 146, pp 863-869). For instance, U.S. Pat. Nos. 5,574,008, 5,512,549, 5,705,483, 5,981,488, 6,191,102 and 6,583,111, which are incorporated herein by reference, disclose compositions of GLP-1s and their clinical use.

In an embodiment, GLP-1s may be used locally at higher than systemic doses, for example, for the treatment of localized hypothermia. glucoprivation in brain or for anesthesia. See for example. Am J. Physiol. Regul. Integr. Comp. Physiol. 292: R1792-R1798, 2007, which is incorporated herein by reference.

In an embodiment, at least one therapeutic agent to treat neurological or behavioral disorders (See FIG. 16 item 772). An example of a short-lived therapeutic agent that might be useful in the treatment of a neurologic disorder such as bipolar disease is venlafaxine (Efexor) 774, which has a half-life of about 5 hours. Neurologic disorders could include, without limitation, several different categories of disorders:
1. Brain tumors: Localized use of short-lived anticancer therapeutic agents to treat brain tumors.
2. Neurodegenerative disorders such as Alzheimer's and Parkinson's disease.
3. Psychiatric disorders: mental depression, obsessive compulsive disorder, bipolar disorder.
4. Behavioral disorders: possibly obesity.
5. Vascular disorders such as stroke.

For example, U.S. Pat. Nos. 6,572,890, 6,703,044 and 7,008,641, which are incorporated herein by reference, disclose formulations and devices for treatment with venlafaxine.

In an embodiment, venlafaxine may be useful as a localized brain stimulant.

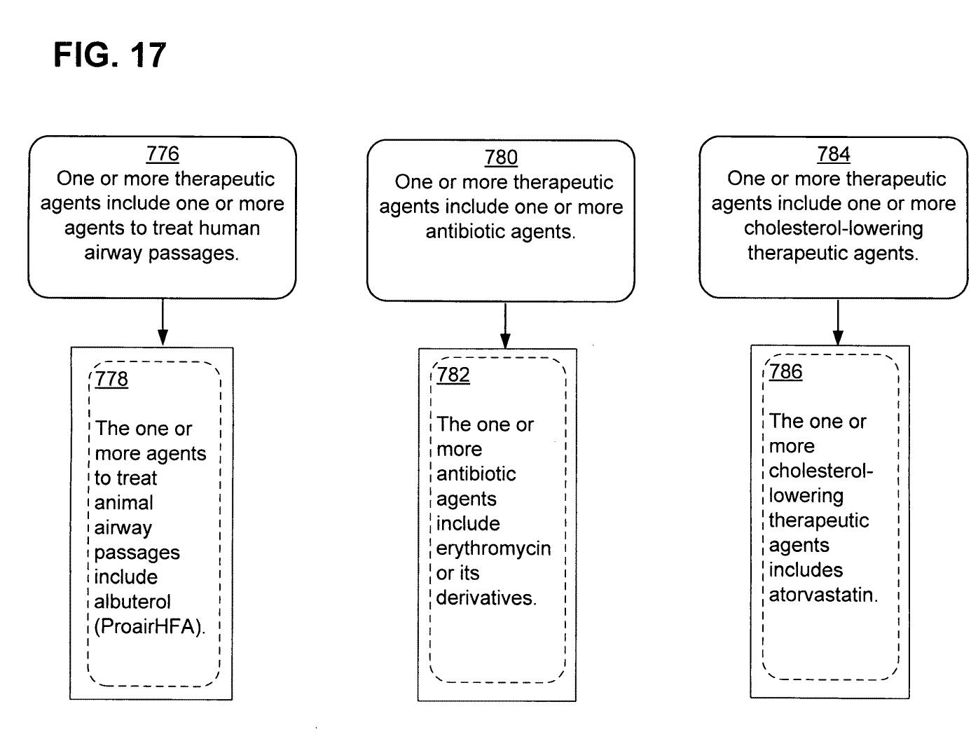

At least one therapeutic agent includes at least one agent to treat human airway passages 776 (FIG. 17). An example of a therapeutic agent useful in this context is albuterol sulfate 778, which has a reported half-life of about 5 hours. Bronchiodilators of the albuterol class may be used systemically as a syrup or tablets or as inhalers (ProairHFA).

Localized infusion at non-systemic doses for acute clinical indications in a patient's airway may be a clinical application of albuterol and its analogs. Examples of this treatment approach may include, without limitation, treatment of acute conditions, such as pneumonia or other respiratory conditions, using localized infusion with intubation devices. Other examples of localized infusion/diffusion may include treatment of lung cancer (See Lung Cancer, Vol. 55, pp 241-247, which is incorporated herein by reference).

For example, U.S. Pat. Nos. 4,499,108, 5,362,755, 5,662,933, 5,958,456 and 6,131,566, which are incorporated herein by reference, disclose methods of formulation, and methods for controlled release of albuterol and its derivatives for clinical applications.

In an embodiment, at least one therapeutic agent includes at least one antibiotic agent 780. Examples of short-lived antibiotics for localized application at non-systemic concentrations include, but are not limited to, erythromycin or its derivatives 782. Erythromycin has a half-life of approximately 1.5 hours.

Erythromycin has an antimicrobial spectrum similar to or slightly wider than that of penicillin, and is often used for patients who have an allergy to penicillins. Erythromycin is also indicated for respiratory tract infections.

For instance, U.S. Pat. Nos. 2,653,899, 4,331,803, 4,349,545, 4,496,717, 5,444,051, 4,990,602, 5,824,513 and 6,162,793, which are incorporated herein by reference, disclose methods of preparation of erythromycin and its derivatives and clinical uses thereof.

In an embodiment, erythromycin or its analogues may be used in higher-than-systemic doses for indications of acute and localized microbial infections. For example, erythromycin or its analogues may be delivered locally upstream of an infection.

In an embodiment, at least one therapeutic agent includes at least one cholesterol-lowering therapeutic agent 784. The at least one cholesterol-lowering therapeutic agent includes, but is not limited to, statins 786. Most statins have long half-lives (around 14-20 hours) with the exception of atorvastatin, which has a half-life of about 2 hours. Thus atorvastatin may be useful as a therapeutic agent for localized clinical applications at higher than systemic dosages. Clinical applications may include, but are not limited to, acute cases of cholesterol-related disease. Localized administration of statins in patients with acute cases of thrombolytic plaques, without limitation, may be an example for the localized use of statins. For example, see Circulation, vol. 99, pp 185-188 (1999) and Pharmacy World and Science, Vol. 23, pp. 177-178 (2001), which are incorporated herein by reference.

Examples of U.S. Pat. Nos. 6,159,997, 6,605,728, 6,605,636, 6,600,051, 6,750,353 and 6,455,574, which are incorporated herein by reference, disclose production methods and potential uses of atorvastatin.

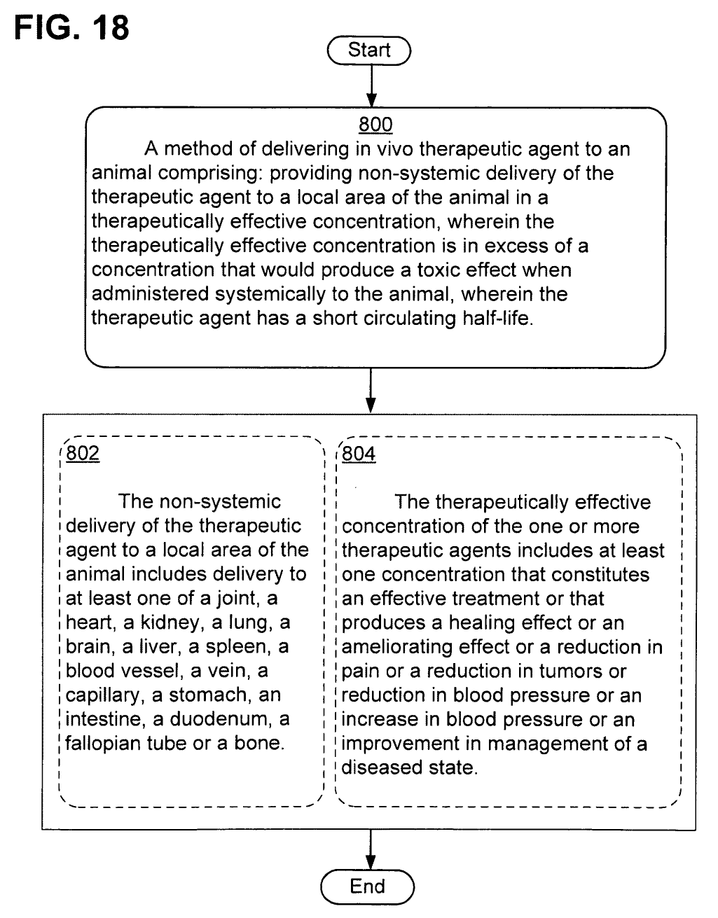

An embodiment includes a method of delivering in vivo a therapeutic agent to an animal (See FIG. 18). The method comprises the following steps: the step 800 of providing non-systemic delivery of the therapeutic agent to a local area of the animal in a therapeutically-effective concentration, wherein the therapeutically-effective concentration is in excess of a concentration that would produce a toxic effect when administered systemically to the animal. Additionally, the therapeutic agent has a short half-life.

Furthermore, a delivery method optionally includes a step 802 of non-systemically delivering a therapeutic agent to a local area of the animal. In an embodiment, the delivery to the local area includes, but is not limited to, delivery to at least one of a joint, a heart, a kidney, a lung, a brain, a liver, a spleen, a blood vessel, a vein, a capillary, a stomach, an intestine, a duodenum, a fallopian tube, a limb appendage, non-vasculaturized area or a bone. Additionally or alternatively, the delivery method includes a step 804 of delivering at least one concentration of at least one therapeutic agent that constitutes an effective treatment or that produces at least one of a healing effect, an ameliorating effect, a reduction in pain, a reduction in tumors, reduction in blood pressure, an increase in blood pressure or an improvement in management of a diseased state.

Software may be used in performing a variety of the methods as described herein. Such software includes software for controlling delivery of a material from a delivery device, including instructions for generating an electromagnetic control signal including frequency components absorbable by at least one controllable output mechanism of a delivery device in an environment, the delivery device including a deformable reservoir capable of receiving and containing a delivery material, and having at least one outlet, wherein the delivery of the material is controllable by the at least one controllable output mechanism; and instructions for controlling the transmission of the electromagnetic control signal to the delivery device with signal characteristics sufficient to activate the at least one controllable output mechanism in the delivery device to control the delivery of material in the delivery device.

The software may include instructions for generating the electromagnetic control signal and include instructions for calculating the electromagnetic control signal based on a model. The instructions for generating the electromagnetic control signal may include instructions for generating the electromagnetic control signal based on a pattern stored in a data storage location, or instructions for generating the electromagnetic control signal based upon a feedback control algorithm. For example, the instructions for generating the electromagnetic control signal may include instructions for generating the electromagnetic control signal based upon a variable feedback control algorithm. The software may include instructions for receiving a feedback signal corresponding to at least one parameters sensed from the environment; and instructions for generating the electromagnetic control signal based at least in part upon the received feedback signal, the electromagnetic control signal having signal characteristics expected to produce a desired feedback signal. Some embodiments of the software may include instructions for receiving a feedback signal from the delivery device; and instructions for generating the electromagnetic control signal based at least in part on the received feedback signal, the electromagnetic control signal having frequency composition and amplitude expected to produce a desired feedback signal. In some embodiments, the software may include instructions for receiving user input of at least one control parameters; and instructions for generating the electromagnetic control signal based at least in part upon the at least one control parameters. In some embodiments, the software may include instructions for performing encryption of the electromagnetic control signal. Instruction may be included for performing an authentication procedure between a remote controller transmitting the electromagnetic control signal and a delivery device including the controllable output mechanism intended to be activated by the electromagnetic control signal. At least a portion of the instructions generating the electromagnetic control signal and the instruction for controlling the transmission of the electromagnetic control signal are executable in distributed fashion on a plurality of microprocessors. Some embodiments of the software may include channel allocation instructions configured to control the allocation of control signal transmission channels for transmission of a plurality of control signals to a corresponding plurality of delivery devices.

With regard to the hardware and/or software used in the control of devices and systems according to the embodiments described herein, and particularly to the sensing, analysis, and control aspects of such systems, those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency or implementation convenience tradeoffs. Those having skill in the art will appreciate that there are various vehicles by which processes and/or systems described herein can be effected (e.g., hardware, software, and/or firmware), and that the preferred vehicle will vary with the context in which the processes are deployed. For example, if an implementer determines that speed and accuracy are paramount, the implementer may opt for a hardware and/or firmware vehicle; alternatively, if flexibility is paramount, the implementer may opt for a solely software implementation; or, yet again alternatively, the implementer may opt for some combination of hardware, software, and/or firmware. Hence, there are several possible vehicles by which the processes described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary.

The foregoing detailed description has set forth various embodiments of the devices and related processes or methods via the use of block diagrams, flowcharts, and/or examples. Insofar as such block diagrams, flowcharts, and/or examples contain at least one function and/or operation, it will be implicitly understood by those with skill in the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in standard integrated circuits, as at least one computer programs running on at least one computer (e.g., as at least one programs running on at least one computer systems), as at least one programs running on at least one processor (e.g., as at least one program running on at least one microprocessor), as firmware, or as virtually any combination thereof, and that designing the circuitry and/or writing the code for the software and/or firmware would be well within the capabilities of one of skill in the art in light of this disclosure. In addition, those skilled in the art will appreciate that certain mechanisms of the subject matter described herein are capable of being distributed as a program product in a variety of forms, and that an illustrative embodiment of the subject matter described herein applies equally regardless of the particular type of signal bearing media used to actually carry out the distribution. Examples of a signal bearing media include, but are not limited to, the following: recordable type media such as floppy disks, hard disk drives, CD ROMs, digital tape, and computer memory; and transmission type media such as digital and analog communication links using TDM or IP based communication links (e.g., links carrying packetized data).

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment).

Those skilled in the art will recognize that it is common within the art to describe devices for detection or sensing, signal processing, and device control in the fashion set forth herein, and thereafter use standard engineering practices to integrate such described devices and/or processes into fluid handling and/or delivery systems as exemplified herein. That is, at least a portion of the devices and/or processes described herein can be integrated into a fluid handling and/or delivery system via a reasonable amount of experimentation.

Those having skill in the art will recognize that systems as described herein may include at least one of a memory such as volatile and non-volatile memory, processors such as microprocessors and digital signal processors, computational-supporting or -associated entities such as operating systems, user interfaces, drivers, sensors, actuators, applications programs, at least one interaction devices, such as data ports, control systems including feedback loops and control implementing actuators (e.g., devices for sensing osmolality, pH, pressure, temperature, or chemical concentration, signal generators for generating electromagnetic control signals). A system may be implemented utilizing any suitable available components, combined with standard engineering practices.

The foregoing-described aspects depict different components contained within, or connected with, different other components. It is to be understood that such depicted architectures are merely examples and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” “operably linked” or “operably coupled,” to each other to achieve the desired functionality.

While particular aspects of the present subject matter described herein have been shown and described, it will be obvious to those skilled in the art that, based upon the teachings herein, changes and modifications may be made without departing from this subject matter described herein and its broader aspects and, therefore, the appended claims are to encompass within their scope all such changes and modifications as are within the true spirit and scope of this subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “the at least one” to introduce claim recitations. However, the use of such phrases should NOT be construed to imply that the introduction of a claim recitation by the indefinite articles “a” or “an” limits any particular claim containing such introduced claim recitation to inventions containing only one such recitation, even when the same claim includes the introductory phrases “at least one” or “at least one” and indefinite articles such as “a” or “an” (e.g., “a” and/or “an” should typically be interpreted to mean “at least one” and/or “at least one”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense of one having skill in the art would understand the convention (e.g., “a system having at least one of A, B, and C” would include but not be limited to systems that have A alone, B alone, C alone, A and B together, A and C together, B and C together, and/or A, B, and C together). In those instances where a convention analogous to “at least one of A, B, or C, etc.” is used, in general such a construction is intended in the sense of one having skill in the art would understand the convention (e.g., “a system having at least one of A, B, or C” would include but not be limited to systems that have A alone, B alone, C alone, A and B together, A and C together, B and C together, and/or A, B, and C together).

Although the methods, devices, systems and approaches herein have been described with reference to certain preferred embodiments, other embodiments are possible. As illustrated by the foregoing examples, various choices of remote controller, system configuration and fluid handling/delivery device may be within the scope of the invention. As has been discussed, the choice of system configuration may depend on the intended application of the system, the environment in which the system is used, cost, personal preference or other factors. System design, manufacture, and control processes may be modified to take into account choices of use environment and intended application, and such modifications, as known to those of skill in the arts of device design and construction, may fall within the scope of the invention. Therefore, the full spirit or scope of the invention is defined by the appended claims and is not to be limited to the specific embodiments described herein.

While various aspects and embodiments have been disclosed herein, other aspects and embodiments will be apparent to those skilled in the art. It is intended that the various aspects and embodiments disclosed herein are for purposes of illustration and are not intended to be limiting, with the true scope and spirit being indicated by the following claims.

1. An implantable medical device, comprising:
at least one reservoir that holds at least one therapeutic agent;
a delivery mechanism configured to provide non-systemic delivery of the at least one therapeutic agent to a local area of an animal in a therapeutically-effective concentration;
wherein the therapeutically-effective concentration is in excess of a concentration that would produce a toxic effect if administered systemically to the animal; and
wherein the at least one therapeutic agent has a half-life of about 5 hours or less.

2. The device of claim 1, wherein the at least one therapeutic agent includes anti-coagulants of blood.

3. The device of claim 2, wherein the anti-coagulants of blood include tissue plasminogen activator or heparin.

4. The device of claim 1, wherein the at least one therapeutic agent includes an anti-cancer agent.

5. The device of claim 4, wherein the at least one therapeutic agent is selected from a group consisting of podophyllotoxin, etoposide, chlorambucil, cisplatin, oxaliplatin, carboplatin and OGX-011.

6. The device of claim 1, wherein the at least one therapeutic agent includes an anti-inflammatory agent.

7. The device of claim 1, wherein the at least one therapeutic agent includes at least one agent for gene therapy of diseases.

8. The device of claim 7, wherein the at least one agent for gene therapy includes a RNA, siRNA, RNAi, or DNA.

9. The device of claim 1, wherein the at least one therapeutic agent has a half-life of about 4 hours or less.

10. The device of claim 1, wherein the at least one therapeutic agent has a half-life of about 3 hours or less.

11. The device of claim 1, wherein the at least one therapeutic agent has a half-life of about 2 hours or less.

12. The device of claim 1, wherein the at least one therapeutic agent has a half-life of about 1 hour or less.

13. The device of claim 1, wherein the at least one therapeutic agent includes at least one agent to treat cardiac arrhythmia.

14. The device of claim 13, wherein the at least one agent to treat cardiac arrhythmia is selected from a group consisting of lidocaine, atropine and adenosine.

15. The device of claim 1, wherein the at least one therapeutic agent includes at least one agent to treat a neurological or behavioral disorder.

16. The device of claim 15, wherein the at least one agent to treat a neurological disorder or behavioral disorder includes venlafaxine.

17. The device of claim 1, wherein the at least one therapeutic agent includes at least one agent to treat human airway passages.

18. The device of claim 17, wherein the at least one agent to treat human airway passages includes albuterol.

19. The device of claim 1, wherein the at least one therapeutic agent includes at least one antibiotic agent.

20. The device of claim 19, wherein the at least one antibiotic agent includes erythromycin or its derivatives.

21. The device of claim 1, wherein the at least one therapeutic agent includes at least one cholesterol-lowering therapeutic agent.

22. The device of claim 21, wherein the at least one cholesterol-lowering therapeutic agent includes atorvastatin.

23. The device of claim 1, wherein the at least one reservoir includes one of a lipid bilayer, a microcapsule, a liposome, a granule, capsule or a radioactive seed.

24. The device of claim 1, wherein the implantable device further comprises at least one of a catheter, a radioactive seed, a stent, a needle, a syringe, a trocar, a microchip or a tube.

25-47. (canceled)

48. A system, comprising:
a means for providing non-systemic delivery of at least one therapeutic agent to a local area of an animal in a therapeutically-effective concentration, wherein the therapeutically-effective concentration is in excess of a concentration that would produce a toxic effect if administered systemically to the animal, and wherein the at least one therapeutic agent has a half-life of about 5 hours or less.

49. The system of claim 48, wherein a means for providing a non-systemic delivery of at least one therapeutic agent to a local area of an animal includes delivery to at least one of a joint, a heart, a kidney, a lung, a brain, a liver, a spleen, a blood vessel, a vein, a capillary, a stomach, an intestine, a duodenum, a fallopian tube, non-vasculaturized area or a bone.

50. The system of claim 48, wherein the at least one therapeutic agent includes an anti-coagulant of blood.

51. The system of claim 48, wherein the anti-coagulants of blood include tissue plasminogen activator or heparin.

52. The system of claim 48, wherein the at least one therapeutic agent includes an anti-cancer agent.

53. The system of claim 52, wherein the at least one anti-cancer agent includes podophyllotoxin, etoposide, chlorambucil, cisplatin, oxaliplatin, carboplatin or OGX-011.

54. The system of claim 48, wherein the at least one therapeutic agent includes an anti-inflammatory agent.

55. The system of claim 48, wherein the at least one therapeutic agent has a half-life of about 4 hours or less.

56. The system of claim 48, wherein the at least one therapeutic agent has a half-life of about 3 hours or less.

57. The system of claim 48, wherein the at least one therapeutic agent has a half-life of about 2 hours or less.

58. The system of claim 48, wherein the at least one therapeutic agent has a half-life of about 1 hour or less.

59. The system of claim 48, wherein the at least one therapeutic agent includes at least one agent to treat cardiac arrhythmia.

60. The system of claim 59, wherein the at least one agent to treat cardiac arrhythmia includes lidocaine, atropine or adenosine.

61. The system of claim 48, wherein the at least one therapeutic agent includes at least one agent to treat a neurological disorder or behavioral disorder.

62. The system of claim 61, wherein the at least one agent to treat neurological disorder or behavioral disorder includes venlafaxine.

63. The system of claim 48, wherein the at least one therapeutic agent includes at least one agent to treat human airway passages.

64. The system of claim 63, wherein the at least one agent to treat human airway passages includes albuterol.

65. The system of claim 48, wherein the at least one therapeutic agent includes at least one antibiotic agent.

66. The system of claim 65, wherein the at least one antibiotic agent includes at least one of erythromycin or a derivative thereof.

67. The system of claim 48, wherein the at least one therapeutic agent includes at least one cholesterol-lowering therapeutic agent.

68. The system of claim 67, wherein the at least one cholesterol-lowering therapeutic agent includes atorvastatin.

69-91. (canceled)
